(19) United States
(12) Patent Application Publication
(10) Pub. No.: US 2008/0208101 A1
(43) Pub. Date: Aug. 28, 2008

(54) Diversion Of Emboli During Fluid Circulation

(75) Inventor: Fredrik Boris-Moller, Lund (SE)

Correspondence Address: LADAS & PARRY LLP, 224 SOUTH MICHIGAN AVENUE, SUITE 1600, CHICAGO, IL, 60604, US

(73) Assignee: SIFR 2000 AB, Lund (SE)

(21) Appl. No.: 11/908,819

(22) PCT Filed: Mar. 17, 2006
(86) PCT No.: PCT/SE06/50034

§ 371 Date: Sep. 17, 2007

(30) Foreign Application Priority Data
Mar. 24, 2005 (SE) 0500661-4

(51) Int. Cl. A61M 1/34

(52) U.S. Cl. 604/4.01

(57) ABSTRACT

A method of diversion of an embolus during fluid circulation in an extracorporeal circuit which includes the steps of establishing a first circuit (100, 101a, 101b, 101c, 102, 103, 104, 106b, 106a, 107) between an outlet of a vascular system and an inlet of said vascular system; establishing a 5 second circuit (101b, 101c, 102, 103, 104, 106b, 108) configured to bypass said inlet and outlet; providing a fluid reservoir (102) a pumping means (103), and a bubble detector (105) in said circuits; detecting by means of said bubble detector (105) an embolus in said first circuit and sending a signal to an automatic embolus diversion supervisor (121) connected to said bubble detector (105); and controlling fluid flow direction means (110, 111) in dependence of the signal from said bubble detector (105); whereby said fluid flow direction means (110, 111) are controlled to redirect a detected embolus to said second circuit.

### TECHNICAL FIELD

The present invention relates to diversion of an embolus during fluid circulation. More specifically the present invention relates to the separation of an embolus comprised in a flowing fluid before the flowing fluid reaches a target.

### BACKGROUND OF THE INVENTION

During extracorporeal circulation in e.g. heart surgery there is a risk to introduce detrimental matter into the circulation of the patient. The formation of blood clots and gas bubbles can occur unobtrusively despite rigorous routines to avoid the formation of these emboli, or it can occur from malpractice by the involved personnel. Gas emboli are formed in circulating blood due to cavitation, temperature gradients, and the surplus amount of gases dissolved in blood. In the case the extracorporeal circuit contains a gas-exchange device e.g. an oxygenator there is even a greater risk of gaseous emboli.

Gas emboli can be detected by different techniques, and all modern heart-lung machines are equipped with a warning system to detect gas emboli. Typically the bubble sensor can discern bubbles with a diameter of approximately 0.3 mm, but the main pump is halted first when a bubble with a diameter of 3-5 mm is recognized, as reported in e.g. the manuals of Jostra HLM20 and Stockert S3 heart-lung machines. It is obvious for a person skilled in the art that bubbles or other emboli of millimeter-size may be harmful for the organism since capillaries are much smaller with diameters a hundred times less. Thus, the built-in security measures against bubble injection in heart-lung machines currently on the market seem inadequate. Besides, when an embolus halts the heart-lung machine the patient is deprived completely of circulation and a series of maneuvers has to be performed by the perfusionist and the surgeon to rid the tubing from the embolus. This may take some time and there is a great risk in misunderstandings and mishandling of the situation due to stress. Formerly, extracorporeal procedures were most often performed in hypothermia which increased the acceptable period of no-circulation, but nowadays most procedures are in normothermia, i.e. at normal temperatures, which adds to the stress. Thus, not only the adverse effects of bubbles per se, but also the cumbersome mandatory maneuvers to rid the device of sensed bubbles before their entry into the patient, may be deleterious.

The symptoms in patients appearing after a heart operation with extracorporeal circulation has been termed “Pumphead” and include long lasting and possibly permanent cognitive decline, cf. Scientific American, 2003, (289) 68-73. Furthermore, symptoms also include personality changes, difficulties in concentration and emotional unstableness. Bearing in mind that more than a million extracorporeal procedures are performed in the world annually this is a formidable problem. Researchers in the field speculate that the cause of these adverse effects stems from microembolization and depositing gas or fat into the capillaries of the brain as well as activation of the inflammatory response, complement system etc. Focal neurological deficits also add to postpump complications, and these have been attributed to the manipulation of ascending aorta, letting free solid parts of ateheromatous plaques into the blood stream which subsequently obstruct cerebral vessels.

During heart operations with extracorporeal circulation, the tube supplying oxygenated blood to the body is introduced into the circulation through an aortic cannula introduced into the ascending aorta. Thus large bubbles may enter the circulation at a location where they are immediately directed into the cerebral circulation. The jet of blood entering the ascending aorta is concentrated in size and of high speed and may therefore preferentially enter one of the carotid arteries. The capillary bed of the brain serves as a filter for the blood from the heart-lung machine, which is disadvantageous and at least in part may explain the frequent clinical neurological deficiencies encountered after heart surgery.

In the prior art, several methods focus on eliminating bubbles from circulating fluids including blood. An example of how to divert bubbles from an extracorporeal circuit is presented in U.S. Pat. No. 6,478,962, wherein bubbles are separated by radial centrifugal forces in a chamber constructed for the purpose which is incorporated into the extracorporeal circuit. The U.S. Pat. No. 6,053,967 discloses a method based on a similar principle, and in the U.S. Pat. No. 6,328,789 bubbles are separated by gravitational force. Also the method presented in the international patent application WO 02/072236 relies on the differences in density of the liquid and comprising emboli. None of these methods have means to detect bubbles in the blood stream when they occur. They all separate emboli mechanically due the difference in density of the emboli and the surrounding liquid. Also, in catastrophic events, when large amounts of air enter the system, these methods easily get saturated, letting air through into the blood vessels of the patient.

### PURPOSE OF THE INVENTION

The purpose of the present invention is to provide a method, an apparatus and a system for the elimination of an embolus, such as a gas bubble, in blood in an extracorporeal circulation circuit. Also very large amounts of gas emboli will be able to be diverted from entering the circulation of the patient. The automatic embolus diversion design of the present invention enables the device to decrease the total embolic load during the procedure. This is accomplished by constantly choosing the smallest possible sensed embolus to be diverted from the patient without adversely interfering with the perfusion. Also, automation will accomplish that the surgeon will not be disturbed in situations where today his attention to help debubble the tubing system is needed.

### BRIEF DESCRIPTION OF THE INVENTION

The problem to avoid an inadvertent introduction of gas bubbles or other emboli into the organism is solved according to the invention by a method, an apparatus and a system according to the independent claims. Preferred embodiments of the invention are set out in the dependent claims.

The invention utilizes the signal from a bubble detector to automatically activate or deactivate a system of clamps or valves attached to an arterial line used for the extracorporeal circulation. Thus, a gas bubble detected in the arterial line is directed away from the patient via an arterio-venous tubing to the venous line of the extracorporeal system. This is accomplished by the action of a control unit that has the ability to control and to calculate timing of the clamping and declamping of the arterio-venous connecting tube. This is performed concomitantly as the aortic cannula is clamped during the period of time that the bubble is redirected over to the venous line via the arterio-venous tubing. Immediately after the bubble has passed, the clamps are to be reset to resume blood flow into the patient again. This sequence of exactly timed actions of different parts of the automatic device will need a computer designed for the purpose. It may need inputs from the heart-lung machine regarding current delivery rate of blood and also size of current tube diameter and distance of the arterial line from the sensor to the diverting valve. Also time constants of all parts of the automatic device have to be accounted for by the computer.

The present invention will be described with reference to extracorporeal circulation in for example heart surgery, but other embodiments are obvious for the person skilled in the art of perfusion and the invention can also be used in other clinical applications, such as in the clinical setup for dialysis. Also in industrial processes the design of this invention may be useful.

### DESCRIPTION OF THE DRAWINGS

The present invention will be described in further detail below, with reference to the accompanying drawings, of which

FIG. 1 illustrates the state of art for extracorporeal circulation in heart surgery. FIG. 1a shows the tubing in a majority of cases. FIG. 1b utilizes an additional arterio-venous tubing connection close to the patient, operated by the thoracic surgeon while initiating extracorporeal circulation and weaning from the pump.

FIG. 2 shows schematically a first embodiment of the inventive system;

FIG. 3 shows schematically a detailed illustration of the extracorporeal tubing system close to the patient in accordance with the first embodiment of the invention as depicted in FIG. 2;

FIG. 4 is a schematic illustration of how a method for in vitro testing of the system can be performed before initiation of extracorporeal circulation;

FIG. 5 shows schematically an alternatively constructed extracorporeal part of the first embodiment of the present invention;

FIG. 6 shows schematically a second embodiment of the inventive system;

FIG. 7 shows schematically an enlarged view of the automatic embolus diversion supervisor according to the invention; and

FIG. 8 depicts schematically a third embodiment of the inventive system, intended for dialysis.

### DETAILED DESCRIPTION OF THE INVENTION

The present invention relates to control and diversion of bubbles in tubings where liquid is propelled. More specifically the present invention refers to simple, quick and efficient avoidance of bubbles entering into the circulation of a living being. This is accomplished by the construction of extracorporeal tubings equipped with devices intended to make it possible to momentarily divert bubbles or emboli, and which is controlled automatically by signals from one or more bubble/emboli sensors. The signals can be analyzed and processed in a computerized device having a central processor. The analyzed and/or processed information can then be utilized for diverting detected emboli and thereby avoiding infusion of hazardous emboli in a patient. The computer can also get input data from a heart-lung machine.

The present invention relates to a system, an apparatus, and a method for controlling the bubbles in an extracorporeal circulatory procedure. It is intended for heart surgery, but can also be employed in a multitude of clinical applications, e.g. dialysis, in which it is desirable to extracorporeally circulate a body fluid.

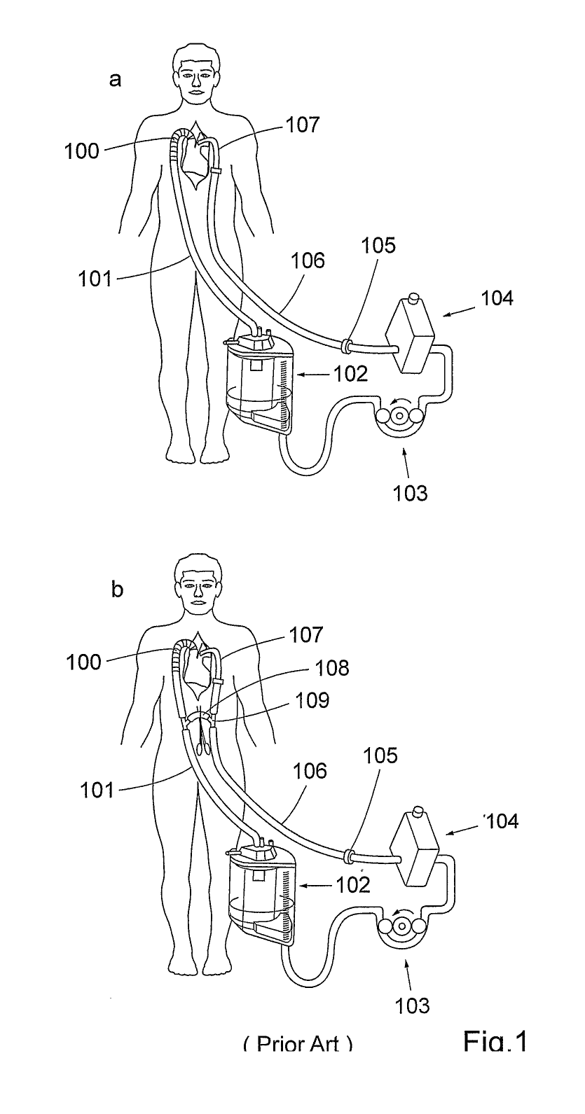

FIG. 1 illustrates schematically the current state of art for the setup of the tubing of the main pump in extracorporeal circulation during open heart surgery. In FIG. 1a venous blood is diverted from vena cava through a venous cannula 100 to a venous tubing or line 101 and enters a venous blood reservoir 102. Here it is filtered and large bubbles may be eliminated by gravitational force. A main pump 103, here shown of a roller pump model, subsequently generates movement energy to the blood which then enters an oxygenator 104 where gas exchange occurs. A bubble detector 105 is attached to an arterial tubing or line 106. In some cases this is done after an optional embolus filter. Blood then enters the blood vessels of the patient through an arterial cannula 107 which in the exemplifying embodiment is introduced into the ascending aorta.

FIG. 1b shows a slightly different setup with an arterio-venous connection 108 of the tubing close to the patient, which is manipulated by the surgeon during the operation. This can be used for the continuous circulation of priming fluid and bubble elimination after cannulation but before the initiation of extracorporeal circulation. Also, during extracorporeal assist, in case an air embolus has halted the main pump 103 by the bubble sensing device 105, the embolus can be diverted from entering into the patient by alerting the surgeon to momentarily open the arterio-venous connection and concomitantly clamp the arterial and venous cannula.

The present invention will now be described in more detail. In the figures the same reference numerals is used for the same or similar features.

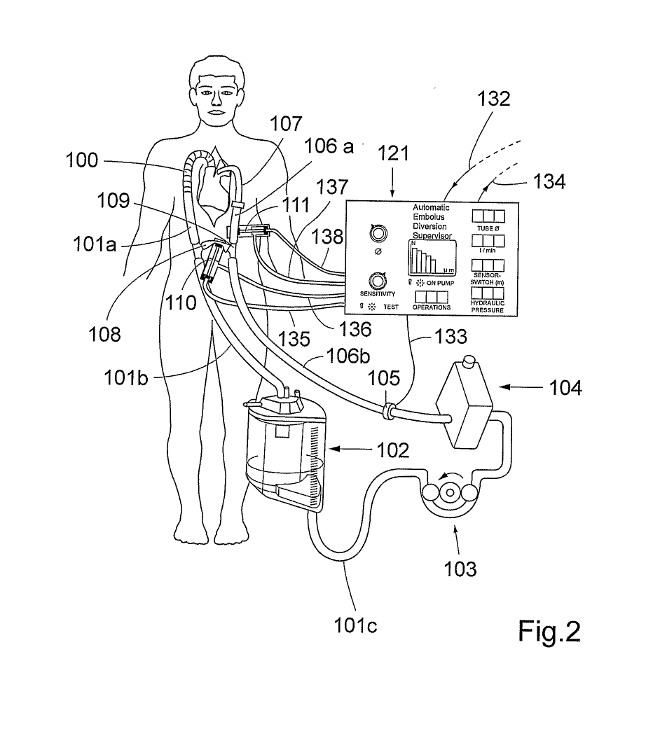

FIG. 2 shows schematically a first embodiment of the present invention. A first fluid circuit 100, 101a, 101b, 101c, 102, 103, 104, 106a, 106b, 107, 109 is established between an outlet from a vascular system of a living being and an inlet into said vascular system. As exemplified in FIG. 2, the outlet is realized as an outlet from the vena cava and the heart and the inlet is realized as an inlet into ascending aorta of the patient. Blood can be extracted from the outlet by means of a extraction means, e.g. a cannula, 100 and extracorporeally circulated in said fluid circuit between the outlet and the inlet.

A second fluid circuit 101b, 101c, 102, 103, 104, 106b, 108, 109 is configured to bypass the inlet and the outlet of the vascular system. As shown in FIG. 2, the body will thus be bypassed by the second fluid circuit. However, it should be understood that the inlet and outlet do not have to be an inlet and an outlet of the same organ or body part, but they can be an inlet and an outlet of different organs or body parts.

A fluid reservoir 102, a pumping means 103, and a bubble detector 105 is arranged at the first and second fluid circuits. The fluid reservoir 102 is also configured to filter the fluid and to, by means of gravitational force, eliminate or reduce large bubbles. The pumping means 103, e.g. a roller pump, is configured to generate movement energy to the fluid in the fluid circuit. The bubble detector 105 is configured to detect an embolus in the first fluid circuit and to send a signal to an automatic embolus diversion supervisor 121 connected to the bubble detector 105. By means of the signal, the automatic embolus diversion supervisor 121 is informed that an embolus is detected. Based on the signal, the automatic embolus diversion supervisor 121 can determine when the fluid flow is to be redirected from the first fluid circuit to the second fluid circuit in order to divert said detected embolus from the inlet, i.e. in order not to introduce the detected embolus into the body part of the living being. When the embolus has passed by a branching element 109 of the fluid circuit, the fluid flow is redirected from the second fluid circuit back to the first fluid circuit.

The automatic embolus diversion supervisor 121 is connected to the extracorporeal tubings 106a, 106b, 108, and to the bubble detector or sensor 105. Further, the automatic embolus diversion supervisor 121 is attached with hydraulic/pneumatic tubes 135, 136, 137, 138 or electric cables (not shown) to the fluid flow redirection means 110, 111. The fluid flow redirection means 110, 111 can be realized as automatic clamping devices 110, 111. Signals from the bubble sensor 105 to the automatic embolus diversion supervisor 121 are transmitted by means of the cable 133 and inputs from and outputs to a heart-lung machine can be transmitted by means of cables 132 and 134. The automatic embolus diversion system 121 is thus configured to control the redirectional valves or clamping devices, to sense and ascertain that redirection of an embolus has occurred, and possibly also to control the pumping means. Further, the automatic embolus diversion system 121 can be configured to display the amount of emboli sensed and redirected.

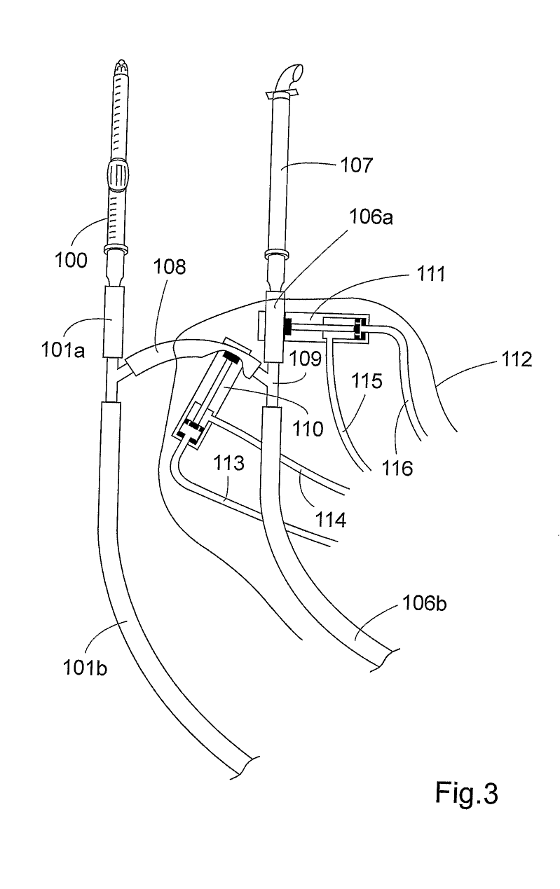

FIG. 3 depicts a part of the extracorporeal tubing system of the first embodiment of the inventive system. In this embodiment the controlling of flow throw the tubing is performed by hydraulic or pneumatic forces working on pistons in cylinders. A vital part of the tubing is the branching element, y-piece, 109 inserted into the arterial line 106a, 106b. Here is where the embolus shall be directed from entering the vascular system of the patient and redirected into the extracorporeal arterio-venous tubing 108. These parts of the tubing are close to the operating field and consequently sterile. The cylinders and pistons 110, 111 are intended for use a multitude of times and therefore not sterile. To be able to attach them to the tubing close to the y-piece 109, this has to be performed while protecting the sterile vicinity from contamination. Therefore the single-use set of tubing could be equipped with a sterile protective shield 112 covering the arterial line 106, the y-piece 109 and a further distance of the connecting arterio-venous tubing 108 so that the cylinders and pistons with their tubing can be attached properly, leaving so much space that in case of malfunction of the device its detaching and the manual clamping and declamping necessary for a safe extracorporeal procedure can easily be performed.

A signal generated by the bubble sensor pumps fluid/gas in the tubes 113, 114 into the device 110 so that the arterio-venous tube 108 is opened for the passage of the embolus. Concomitantly, or shortly afterwards, the arterial flow into the patient through the arterial cannula 107 is halted in a similar but opposite way by pumping fluid/gas in the tubes 115, 116 into the device 111. After an appropriate time period to let the embolus pass into the venous system, the tubing into the patient is reopened and the arterio-venous connection closed.

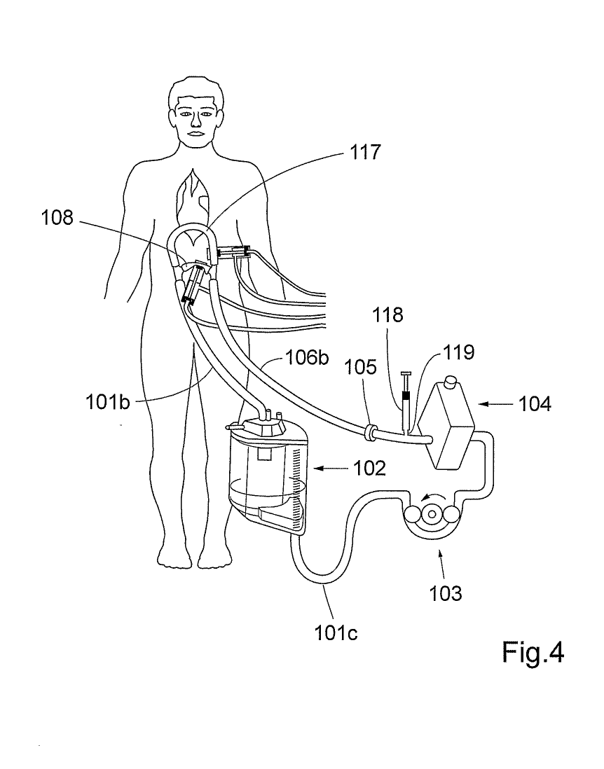

FIG. 4 illustrates the function test of the method. This can be performed in vitro before the commencement of the in vivo procedure. The cannula loop 117, equivalent to 101a and 106a, of the tubing is kept undivided and the automatic embolus diversion system 121 is mounted and activated while the heart-lung machine circulates the priming fluid through 108. A syringe 118 filled with air is attached to the tubing through a conventional Luer-Lock 119 before the embolus sensor 105. Coordinated in time with the surgeon, the perfusionist injects an air embolus from the syringe and the surgeon confirms that the mechanism of the pistons and cylinders are functioning so that the bubble is redirected from the cannula loop 117 into the arterio-venous tubing 108. The testing can be performed at different pump-flows to check for proper calculation algorithm of the automatic embolus diversion supervisor 121.

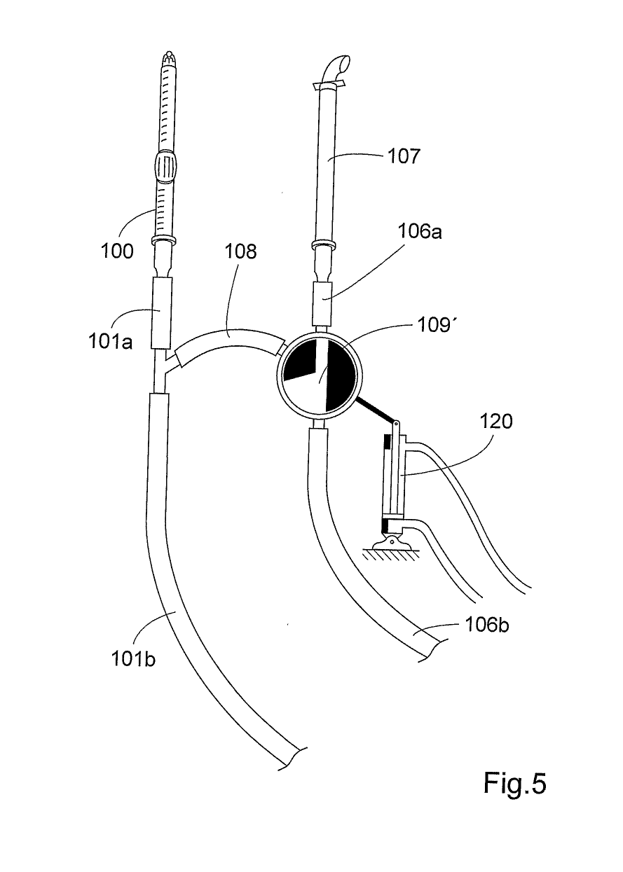

Another embodiment of a part of the extracorporeal tubings is depicted in FIG. 5. In this embodiment, the branching element is realized as a circular, three-ways, valve 109′ which can be controlled by a valve control means 120, e.g. by the position of a piston in a cylinder 120. The cylinder 120 is connected to the valve 109′ and to the automatic embolus diversion supervisor 121.

It is obvious to a person skilled in the art that the invention may be modified in other ways within the scope of the appended claims, thus the mechanism for redirection of emboli may rest upon e.g. mechanic, hydraulic, pneumatic or electromagnetic force.

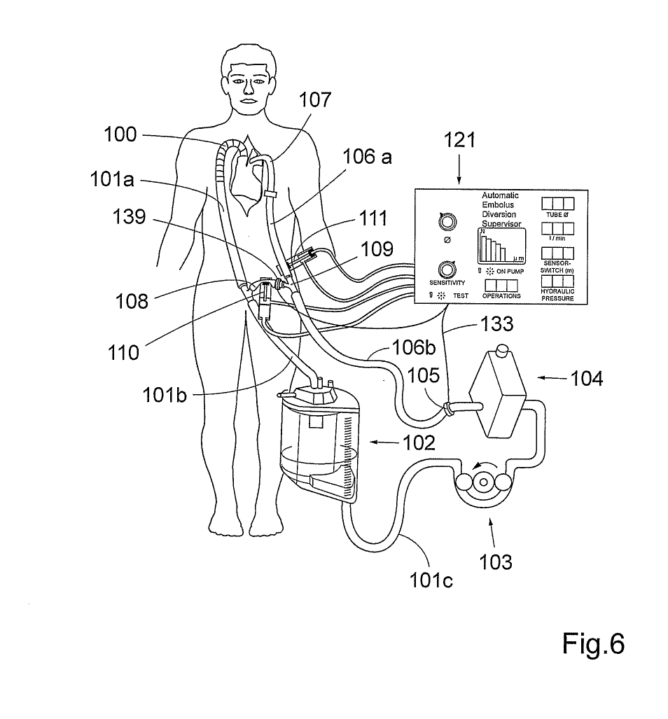

A second embodiment for the additional safety of the inventive system is depicted in FIG. 6 and includes two or more embolus sensors. One of these bubble detectors 105 is attached to the arterial line 106b as shown in the first embodiment. Another detector 139 may be attached to the arterio-venous tubing 108 as shown in FIG. 6. By this arrangement it is possible to ascertain that an embolus in the arterial line 106b, detected by the first sensor 105, indeed has been redirected by the embolus diversion system 121, since, if the embolus has been correctly redirected the second bubble detector 139 will subsequently detect the diverted embolus. Another bubble detector (not shown) may be attached to the arterial line 106a. This arrangement may even be deemed mandatory, since an embolus that ordinarily should halt the main pump, by the present invention does not halt the pump but is redirected. Thus, it is desirable, or mandatory, to ascertain, closer to the patient, the absence of the embolus detected by the first sensor 105, signifying adequate function of the embolus diversion system 121. And, in case the embolus again appears at the sensor closer to the patient, there is still time and space to halt the pump and avoid embolus entry into the vascular system.

This embodiment may need an additional length of the arterial line 106b, since the automatic embolus diversion system 121 will need time, firstly for the redirectional valves or clamping devices to react, secondly for the sensing and ascertaining that redirection of an embolus has occurred, and thirdly, in case of malfunction, for calculation and for the signal to be conveyed to halt the main pump. In FIG. 6 therefore, both the proximal 106a and distal 106b part of the arterial line are shown with increased lengths. In this embodiment the branching element 109, 109′ may need to be incorporated into the tubing so close to the oxygenator 104 that there will be no need for keeping it sterile during the extracorporeal circulatory procedure. With the extra tubing needed for this embodiment extra priming volume may be needed to add. However, if the arterial tubing is of dimension ⅜″, less than 100 ml extra priming volume per meter tube will be satisfactory.

In the case that the automatic embolus diversion system 121 is activated for a prolonged period there may be a need for the temporary closure of the venous line in order not to let an unnecessary amount of blood out from the patient into the venous reservoir. For this purpose, the invention also may include a third automatically controlled cylinder with piston (not shown) attached to the venous cannula 100 or tubing 101a in a similar way as the arterial counterpart 111. However, the necessity to clamp the venous line during a main pump halt is basic to any perfusionist—who is obliged to act in accordance.

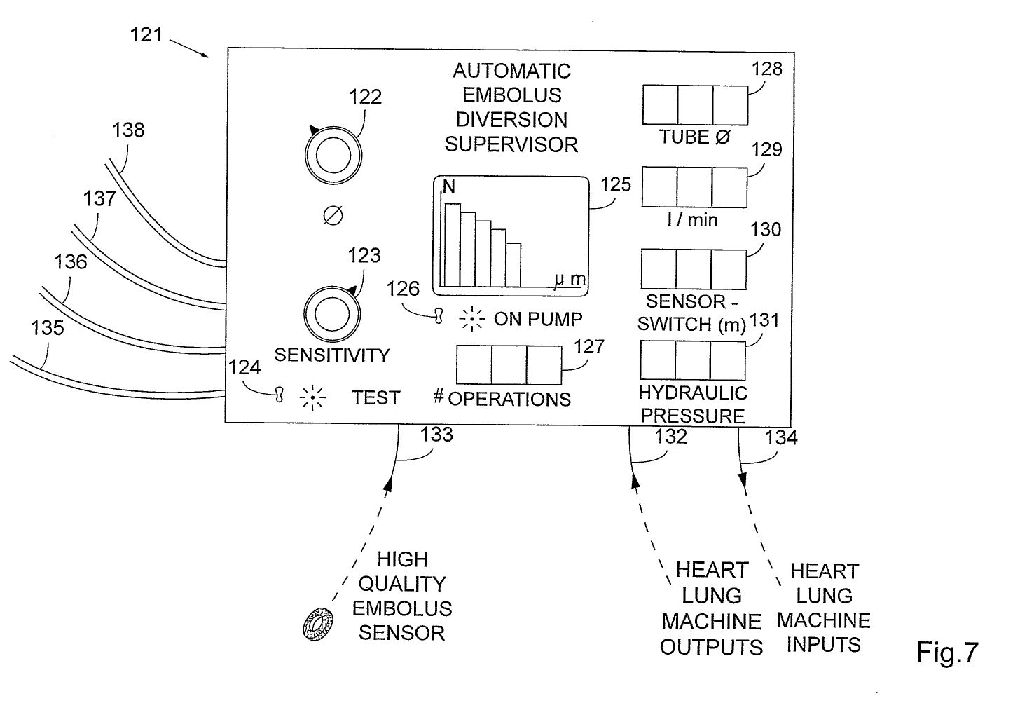

The automatic embolus diversion supervisor 121, see FIG. 7, is the computerized control unit of this invention. The automatic embolus diversion supervisor 121 contains means for proper handling of the system, i.e. interactive means such as displays, knobs, keyboard. The size of the arterial line 106a, 106b diameter can be set by the perfusionist with a knob 122. The sensitivity of the embolus sensor can be chosen with the knob 123, i.e. by means of the knob 123 the diameter of an embolus to be diverted can be set. The knob 123 may also have a position with no sensitivity at all, which position gives that the automatic embolus diversion system is not in operation. The test switch 124 in the on-position makes the automatic embolus diversion supervisor to be activated but not to document an embolus sensed. The screen 125 shows the size and frequency distribution of small emboli that have been sensed but not redirected by the automatic embolus diversion system 121. Thus no emboli larger than the size chosen by the sensitivity knob 123 should be possible to document on the screen 125. Before extracorporeal circulation is commenced, the priming fluid is circulated through the arterio-venous tube 108. Then, when bypass starts the arterio-venous tube 108 is clamped and the arterial 107 and venous 100 cannulae declamped. This maneuver can be performed by the surgeon and perfusionist in concert—the surgeon manually declamps the venous cannula 100 and the perfusionist clamps the arterio-venous tube 108 and declamps the arterial cannula 107 by turning the switch 126 on the display. This may also start the timing of extracorporeal procedure and the documentation of emboli sensing by 105, so that the frequency of operation of the automatic embolus diversion system 121 can be displayed 127 and the sensitivity knob 124 be adjusted in order to achieve optimal performance of the system. Instead of the total number of occasions the automatic embolus diversion system 121 has been activated, the total active time can be shown in this display 127. In this embodiment of the automatic embolus diversion supervisor 121, the displays 128, 129, 130, and 131 show the present setting for some operation parameters, such as arterial line diameter, main pump output, the distance between the sensor 105 and the branching element 109, 109′, and hydraulic pressure, respectively.

The distance between the sensor 105 and the branching element 109, 109′, and the flow rate in the arterial line 106 are important parameters to know in order for the system to bypass a detected embolus optimally in time and thus minimize the amount of blood that is diverted and not reinfused to the patient by means of the arterial cannula 107. During extracorporeal circulation it is desirable to open the flow in the arterio-venous line 108 and to close the flow in the arterial cannula 107 just as a detected embolus enters the branching element 109, 109′ and then, reversely, closing the arterio-venous line 108 and opening the flow towards the arterial cannula 107 just as the entire embolus has passed into the arterio-venous line 108.

Signals of interest from a connected heart-lung machine such as perfusion flow (l/min), bubble detection can be conveyed to the automatic embolus diversion supervisor through the cable 132. Signals from high quality sensor(s) 105 and 139 for emboli detection are transmitted in cable 133. Information and demands from the automatic embolus diversion supervisor 121 to the heart-lung machine are conveyed by the cable 134. Tubes or cables 135, 136, 137, and 138 are for controlling clamping devices.

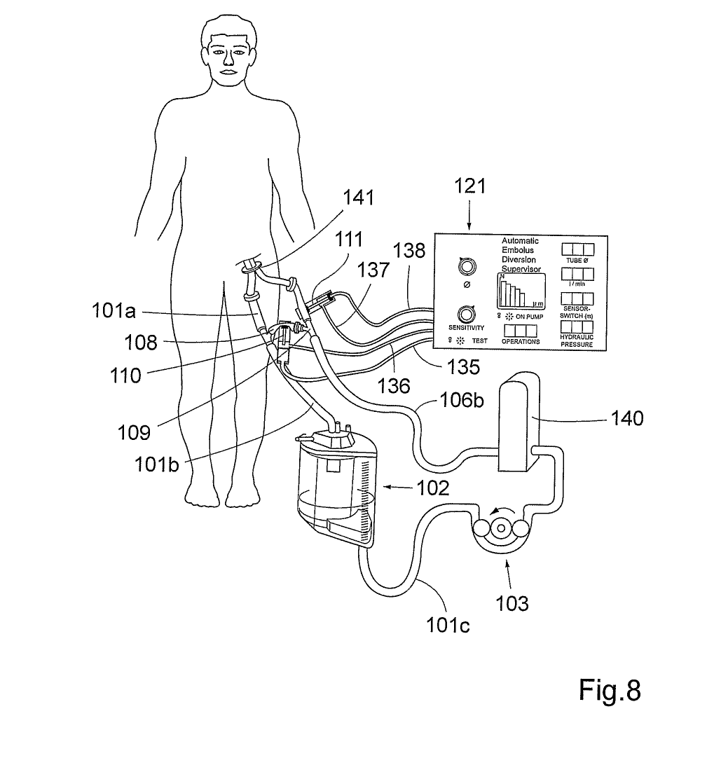

A third embodiment of the inventive system is depicted in FIG. 8. In this application the invention is modified to be used in dialysis. The oxygenator is in this situation not needed and a dialysis membrane 140 is added. In dialysis, the venous reservoir may be of a smaller size as compared to the size of the venous reservoir in the previously described embodiments. However, in case a large bubble has been detected and is redirected according to the method of the invention, the venous reservoir should contain enough volume to minimize operational arrests. In FIG. 8 the dialysis catheter 141 entering the body is depicted as a two-lumen veno-venous dialysis catheter, but other arrangements of cannulation the vascular system for dialysis are feasible.

The present invention has been described above with reference to exemplifying embodiments, and it is obvious to a person skilled in the art that the invention may be modified in other ways within the scope of the appended claims.

1. A method for diversion of an embolus during fluid circulation in an extracorporeal fluid circuit, comprising the steps of:
establishing a first fluid circuit between an outlet from a vascular system of a living being and an inlet into said vascular system, whereby a fluid extracted from said outlet can be extracorporeally circulated in said fluid circuit between said outlet and said inlet;
establishing a second fluid circuit configured to bypass said inlet and outlet of said vascular system;
providing a fluid reservoir, a pumping means, and a bubble detector in said first and second fluid circuits;
detecting by means of said bubble detector an embolus in said first fluid circuit and sending a signal to an automatic embolus diversion supervisor connected to said bubble detector;
determining based on said signal when the fluid flow is to be redirected from said first fluid circuit to said second fluid circuit automatically in order to divert said detected embolus from said inlet; and
automatically redirecting said fluid flow from said first fluid circuit to said second fluid circuit; and when said embolus has passed by a branching element redirecting the fluid flow from said second fluid circuit back to said first fluid circuit.

2. The method of claim 1, further comprising the step of providing fluid flow direction means configured to control the flow through said branching element.

3. The method of claim 2, further comprising the step of attaching the automatic embolus diversion supervisor to said fluid flow direction means and connecting said automatic embolus diversion supervisor to a heart-lung machine comprising an oxygenator or a dialysis machine comprising a dialysis-membrane.

4. The method of claim 2, wherein the fluid flow direction means are realized as automatic clamping devices comprising e.g. cylinders and pistons, and controlled by means of hydraulics, pneumatics, electromagnetic or mechanical springs; and wherein said branching element is realized as a y-connection.

5. The method of claim 1, wherein said branching element is realized as a three-ways valve, the passageway of which is controlled by means of the automatic embolus diversion supervisor via a valve control means.

6. The method of claim 1, further comprising the steps of analyzing the signal received from said embolus detector and calculating when the fluid flow is to be redirected from the first fluid circuit into a tubing of the second fluid circuit automatically in order to bypass said detected embolus.

7. The method of claim 6, further comprising the step of arranging a second and/or third bubble detector at the tubing of the first and/or second fluid circuit close to the branching element, the second and/or third bubble detector being configured to, depending on the size of the bubble, document or shut down a pumping means if it, depending on the location of the sensor(s), does or does not detect a bypassed embolus after a predetermined or calculated period of time.

8. The method of claim 1, further comprising the step of providing means for controlling the opening/closing of valves or clamps in order to minimize the time period needed for safely bypassing a detected embolus.

9. The method of claim 1, further comprising the step of providing interactive means by means of which information may be presented to a user and by means of which a user may select the operation conditions, e.g. the size of the embolus to be redirected.

10. The method claim 2, further comprising the step of providing a sterile protective cover configured to cover the branching element and the fluid flow direction means.

11. A system for diversion of an embolus during fluid circulation in an extracorporeal fluid circuit, comprising means for:
establishing a first fluid circuit between an outlet from a vascular system of a living being and an inlet into said vascular system;
establishing a second fluid circuit configured to bypass said inlet and outlet of;
providing a fluid reservoir, a pumping means, and a bubble detector in said first and second fluid circuits;
said bubble detector being configured to detect an embolus in said first fluid circuit and to send a signal to an automatic embolus diversion supervisor connected to said bubble detector; and
said automatic embolus diversion supervisor is connected to fluid flow direction means arranged at said first and second fluid circuit, respectively, and configured to control said fluid flow direction means in dependence of the signal from said bubble detector; whereby said fluid flow direction means are controlled to redirect a detected embolus to said second fluid circuit.

12. The system of claim 11, wherein the automatic embolus diversion supervisor is attached to the fluid flow direction means by means of hydraulic/pneumatic tubes, electrical cables, radio signals or other means of conveying signals, and wherein said automatic embolus diversion supervisor is connected to a heart-lung machine comprising an oxygenator or to a dialysis machine comprising a dialysis membrane by means of cables.

13. The system of claim 11, wherein the fluid flow direction means are realized as automatic clamping devices comprising e.g. cylinders and pistons, and controlled by means of hydraulics, pneumatics, electromagnetic or mechanical springs; and wherein said branching element is realized as a y-connection.

14. The system of claim 11, wherein said branching element is realized as a three-ways valve, and wherein the automatic embolus diversion supervisor is configured to control the passageway of the valve by means of via a valve control means.

15. The system of claim 11, wherein the automatic embolus diversion supervisor is configured to analyze the signal received from said embolus detector and to calculate when the fluid flow is to be redirected from the first fluid circuit into a tubing of the second fluid circuit in order to bypass said detected embolus.

16. The system of claim 15, wherein a second and/or third bubble detector is arranged at the tubing of the first and/or second fluid circuit close to the branching element, the second and/or third bubble detector being configured to, depending on the size of the bubble, document or shut down a pumping means if it, depending on the location of the sensor(s), does or does not detect a bypassed embolus after a predetermined or calculated period of time.

17. The system of claim 11, wherein the automatic embolus diversion supervisor comprises means for controlling the opening/closing of valves or clamps in order to minimize the time period needed for safely bypassing a detected embolus.

18. The system of claim 11, wherein the automatic embolus diversion supervisor comprises interactive means by means of which information may be presented to a user and by means of which a user may select the operation conditions, e.g. the size of the embolus to be redirected.

19. The system of claim 11, further comprising a sterile protective cover configured to cover the branching element and the fluid flow direction means.

20. An automatic embolus diversion supervisor for diversion of an embolus during fluid circulation in an extracorporeal fluid circuit, wherein said automatic embolus diversion supervisor is configured to be connected to one or more bubble detectors and to receive signals from said bubble detector; said automatic embolus diversion supervisor is further configured to be connected to fluid flow direction means arranged at a first and a second fluid circuit, respectively, and configured to control said fluid flow direction means in dependence of the signal from said bubble detector; whereby said fluid flow direction means are controlled to redirect a detected embolus to said second fluid circuit.

21. The automatic embolus diversion supervisor of claim 20, wherein the automatic embolus diversion supervisor is connected to the fluid flow direction means by means of hydraulic/pneumatic tubes, electrical cables, radio signals or other means of conveying signals, and wherein said automatic embolus diversion supervisor is connected to a heart-lung machine by means of cables, radio signals or other means of conveying signals.

22. The automatic embolus diversion supervisor of claim 20, wherein the automatic embolus diversion supervisor is configured to analyze the signal received from said embolus detector and to calculate when the fluid flow is to be automatically redirected from the first fluid circuit into a tubing of the second fluid circuit in order to bypass said detected embolus.

23. The automatic embolus diversion supervisor of claim 20, wherein the automatic embolus diversion supervisor comprises means for controlling the opening/closing of valves or clamps in order to minimize the time period needed for safely bypassing a detected embolus.

24. The automatic embolus diversion supervisor of claim 20, wherein the automatic embolus diversion supervisor comprises interactive means by means of which information may be presented to a user and by means of which a user may select the operation conditions, e.g. the size of the embolus to be redirected.
